(19) United States
(12) Patent Application Publication
(10) Pub. No.: US 2025/0269315 A1
(43) Pub. Date: Aug. 28, 2025

(54) A PROCESS FOR THE PURIFICATION OF FLUORINATED OLEFINS IN GAS PHASE

(71) Applicant: Solvay SA, Brussels (BE)

(72) Inventors: Joo-Hee Kang, Lyon (FR); Eric Perin, Villefranche Sur Saone (FR); Michael Pittroff, Ahlten (DE); Denis Revelant, Genas (FR)

(73) Assignee: Solvay SA, Brussels (BE)

(21) Appl. No.: 18/247,720

(22) PCT Filed: Sep. 28, 2021
(86) PCT No.: PCT/EP2021/076576

§ 371 Date: Apr. 3, 2023

(30) Foreign Application Priority Data
Oct. 2, 2020 (EP) 20199799.6

(51) Int. Cl. B01D 53/04

(52) U.S. Cl. CPC B01D 53/0415; B01D 2253/106; B01D 2253/308; B01D 2256/26; B01D 2257/206; B01D 2257/80

(57) ABSTRACT

The present invention relates to a process for the purification of fluorinated olefins in gas phase, using at least two adsorbents with different average pore sizes.

The present invention relates to a process for the purification of fluorinated olefins, such as, especially, hexafluoro-1,3-butadiene.

Hexafluoro-1,3-butadiene is a colorless, gaseous unsaturated fluorocarbon with an alternating double bond. It is an etchant showing very high performance for plasma, ion beam, or sputter etching in semiconductor devices manufacturing. Due to its short atmospheric lifetime (<1 day), its negligible global warming potential, and its inertness to the stratospheric ozone layer, hexafluoro-1,3-butadiene is an environmentally compatible gas. Hexafluoro-1,3-butadiene is marketed by Solvay under the brand name Sifren® 46.

Hexafluoro-1,3-butadiene employed in the semiconductor industry must be of extremely high purity. To this end, EP1329442A1 describes a process for the purification of hexafluoro-1,3-butadiene using certain adsorbents with low average pore diameter, particularly molecular sieve 5 Å, since the hexafluoro-1,3-butadiene is apparently excluded from the adsorbent while the impurities are adsorbed and thus, avoiding deleterious decomposition reactions from occurring.

WO2020/164912A1 describes a process for the purification of fluorinated olefins, such as, especially, hexafluoro-1,3-butadiene, using at least two adsorbents having an average pore size of above 6 Å, especially a combination of silica gel and molecular sieve 13X.

However, depending on the initial composition of the crude hexafluoro-1,3-butadiene to be purified, especially the nature and the amount of the impurities present therein, the existing purification processes may be not sufficiently effective. In particular when water is present in the raw hexafluoro-1,3-butadiene to be purified, it may react with certain adsorbents used for the purification thereof and generate thereby new impurities likely to contaminate the final hexafluoro-1,3-butadiene.

Another problem lies in the cost for running adsorbents-based purification processes, especially because certain adsorbents may be quite expensive, and the global cost is all the more increased than it is necessary to replace or regenerate the adsorbents too often.

Additionally, most of the existing purification processes based on the use of adsorbents require a step of activation thereof prior to the purification step, mainly for removing residual moisture. The activation treatment usually consists in a heat treatment at high temperature, typically ranging from 250° C. to 400° C., under a dry inert atmosphere. This step constitutes a supplementary production cost as it wastes energy, time and it requires managing effluents.

Thus, there is still a need for an improved process for the purification of hexafluoro-1,3-butadiene. Consequently, one objective of the present application is to propose an improved process for the purification of hexafluoro-1,3-butadiene, suitable to solve at least one and preferably several of the above mentioned problems. Among others objectives, the present invention aims at providing a fast, simple, economical and/or environment-friendly purification process, which can be run efficiently at industrial scale, as well as providing an hexafluoro-1,3-butadiene having an improved purity, at the very least a purity suitable with electronics applications.

These and other objectives are achieved by the process according to the present invention.

Accordingly, a first aspect of the present invention concerns a process for the purification of hexafluoro-1,3-butadiene comprising a step wherein a gaseous mixture comprising hexafluoro-1,3-butadiene is contacted with at least one first adsorbent and at least one second adsorbent to purify said gas mixture, wherein the at least one first adsorbent has an average pore size of more than 10 Å and the at least one second adsorbent has an average pore size of less than 4 Å. The average pore size may be measured by conventional methods known by a skilled person, in particular by nitrogen adsorption porosimetry.

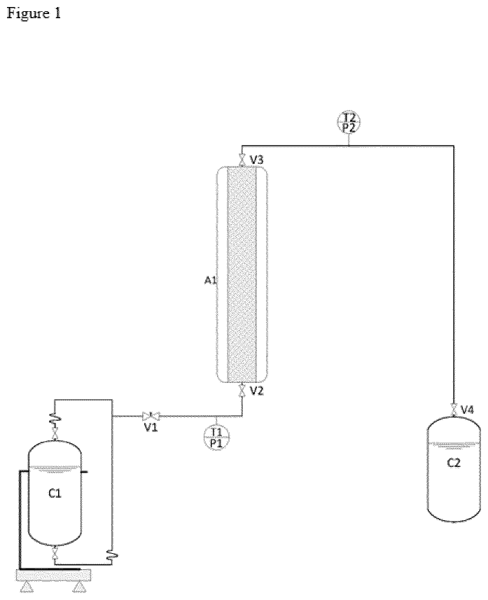

FIG. 1 shows a flow diagram of an apparatus for performing the process according to the present invention.

The gaseous mixture to be purified may contain various impurities in admixture with the hexafluoro-1,3-butadiene, such as water, hydrofluoric acid, hydrohalogenocarbons, especially hydrofluorocarbons and/or hydrochlorofluorocarbons, more particularly hydrohalogenoolefins, especially hydrofluoroolefins and more particularly 1,1,4,4-tetrafluoro-1,3-butadiene or isomers thereof (thereafter referred to as C4H2F4). Said impurities may come from the formation of byproducts, from residual solvents, unreacted starting materials and/or partially unreacted starting materials.

Without being particularly limited, the initial purity of the raw hexafluoro-1,3-butadiene to be purified by the process according to the invention may be equal to or greater than 90% by volume, in particular equal to or greater than 95% by volume, more particularly equal to or greater than 98% by volume, even more particularly equal to or greater than 99% by volume, relatively to the total volume of raw hexafluoro-1,3-butadiene. Especially, the raw hexafluoro-1,3-butadiene may comprise from 0 ppmv to 1500 ppmv, in particular from 5 ppmv to 1000 ppmv of C4H2F4. Especially, the raw hexafluoro-1,3-butadiene may comprise from 0 ppmv to 1500 ppmv, in particular from 8 ppmv to 1000 ppmv of water.

The expression “at least one” in connection with the first or second adsorbent used in the process of the invention means that more than one adsorbent having the required features can be used to purify the gaseous mixture. According to one embodiment, the gaseous mixture comprising hexafluoro-1,3-butadiene is contacted with only one first adsorbent and at least one second adsorbent to purify said gas mixture. According to another embodiment, the gaseous mixture comprising hexafluoro-1,3-butadiene is contacted with at least one first adsorbent and only one second adsorbent to purify said gas mixture. According to another embodiment, the gaseous mixture comprising hexafluoro-1,3-butadiene is contacted with only one first adsorbent and only one second adsorbent to purify said gas mixture.

The at least one first adsorbent is selected from adsorbents having an average pore size of more than 10 Å. In addition to the effectiveness of such an adsorbent for removing various impurities from the raw hexafluoro-1,3-butadiene, water molecules especially, the use thereof may advantageously enable to purify the hexafluoro-1,3-butadiene to a level sufficient to preserve the second adsorbent from a premature degradation, in case the first and second adsorbents are arranged in this order. As the hexafluoro-1,3-butadiene itself is likely to be at least partially adsorbed on the first adsorbent, it is preferable to select a first adsorbent which is inert with regard to hexafluoro-1,3-butadiene, that is to say formed in a material with which hexafluoro-1,3-butadiene will not react. In the framework of the invention, the inertness of the material of the first adsorbent can be highlighted by the absence of formation of “new” impurities in the hexafluoro-1,3-butadiene at the outlet of the first adsorbent and/or the absence of a significant reduction of the yield in purified hexafluoro-1,3-butadiene at the end of the process run (typically not more than 5% reduction). By “new” impurity, it is meant an impurity which was not present in the raw hexafluoro-1,3-butadiene before starting the purification process.

According to one sub-embodiment, the at least one first adsorbent may be selected from adsorbents having an average pore size of more than 10 Å and less than 100 Å, more particularly of more than 10 Å and less than 50 Å and even more particularly of more than 10 Å and less than 20 Å.

According to one embodiment, the at least one first adsorbent is selected from the adsorbents suitable to remove (that is to say to adsorb) at least water. Such a first adsorbent contributes with said at least one second adsorbent to get a final hexafluoro-1,3-butadiene of very high purity. It is also particularly suitable to enhance the lifespan of the at least one second adsorbent in the case the gaseous mixture is first purified with the at least one first adsorbent and subsequently purified with the at least one second adsorbent. Indeed, among the possible impurities present in the raw hexafluoro-1,3-butadiene, water is one of the most prone to react with the second adsorbent. The removal of water prior to the purification operated by the at least one second adsorbent advantageously improves the efficiency of said second adsorbent as it can be exclusively dedicated to the removal of specific organic impurities, such as C4H2F4. In addition, by avoiding a possible reaction of water with the material constituting the second adsorbent, it avoids generating new impurities which would contaminate the final hexafluoro-1,3-butadiene.

Suitable adsorbents that can be used as first adsorbent in the framework of the invention having an average pore size of more than 10 Å include silica gel, zeolite 13X, zeolite MFI, activated alumina, activated carbon and the like. Silica gel is more preferred, especially regarding its cost, its inertness towards hexafluoro-1,3-butadiene and because it advantageously retains water from the raw gas mixture containing the hexafluoro-1,3-butadiene to be purified. A very suitable silica gel includes the Tixosil® range from Solvay as well as SYLOBEAD® SG B125 supplied by Grace.

The at least one second adsorbent that is implemented in the process of the invention is selected from adsorbents having an average pore size of less than 4 Å. The pore size of the second adsorbent may help to get a better selectivity towards certain types of organic impurities which are likely to be present in the hexafluoro-1,3-butadiene to be purified, such as hydrohalogenocarbons, in particular hydrofluorocarbons (HFCs) and/or hydrochlorofluorocarbons (HCFCs), more particularly hydrohalogenoolefins, in particular hydrofluoroolefins (HFOs) and even more particularly 1,1,4,4-tetrafluoro-1,3-butadiene or isomers thereof (C4H2F4). Additionally, it is believed that the average pore size of the second adsorbent is small enough to avoid the adsorption of the hexafluoro-1,3-butadiene itself, which avoids side reactions with said second adsorbent and consequently the formation of further impurities.

According to one sub-embodiment, the at least one second adsorbent may be selected from adsorbents having an average pore size of more than 1 Å and less than 4 Å, in particular of more than 2 Å and less than 4 Å and more particularly of more than 3 Å and less than 4 Å.

According to one embodiment, the at least one second adsorbent is selected from the adsorbents suitable to remove (that is to say to adsorb) at least one impurity selected from hydrohalogenocarbons, more particularly selected from hydrofluorocarbons (HFCs) and/or hydrochlorofluorocarbons (HCFCs), more particularly selected from hydrohalogenoolefins in particular from hydrofluoroolefins (HFOs), even more particularly from 1,1,4,4-tetrafluoro-1,3-butadiene and isomers thereof (C4H2F4).

Suitable adsorbents having an average pore size of less than 4 Å that can be used as second adsorbent in the framework of the invention include zeolites having 8-membered-ring pores. More particularly, mention can be made of Zeolite P, Gmelinite, synthetic Chabazite (SSZ-13, SSZ-62), and the like. Synthetic Chabazite is preferred, especially regarding its selectivity towards 1,1,4,4-tetrafluoro-1,3-butadiene (C4H2F4) and isomers thereof, which are the main organic impurities likely to be present in the hexafluoro-1,3-butadiene to be purified. A very suitable Chabazite includes the HCZC S (H-form) from CLARIANT. Any mention of “Chabazite” in the following description refers to synthetic Chabazite.

According to one embodiment, the gaseous mixture is first purified with the at least one first adsorbent and subsequently purified with the at least one second adsorbent. This embodiment is particularly suitable to improve the lifespan of the second adsorbent; it makes possible to reduce the frequency of its replacement or its regeneration operation.

The final purity of the hexafluoro-1,3-butadiene achieved by the process according to the invention is equal to or greater than 99.9% by volume, preferably equal to or greater than 99.95% by volume, more preferably equal to or greater than 99.98% by volume, and most preferably equal to or greater than 99.99% by volume.

The total amount of water potentially remaining in the purified hexafluoro-1,3-butadiene may be lower or equal to 200 ppmv, in particular lower or equal to 160 ppmv, in particular lower or equal to 80 ppmv, in particular lower or equal to 15 ppmv, in particular lower or equal to 8 ppmv. The total amount of water potentially remaining in the purified hexafluoro-1,3-butadiene may be equal to or greater than 0 ppmv, 0.001 ppmv, in particular equal to or greater than 0.1 ppmv, in particular equal to or greater than 1 ppmv. It may be measured by laser diode spectroscopy or gas chromatography (GC).

The total amount of hydrofluorocarbons potentially remaining in the purified hexafluoro-1,3-butadiene may be lower or equal to 500 ppmv, in particular lower or equal to 300 ppmv, in particular lower or equal to 200 ppmv, in particular lower or equal to 150 ppmv, in particular lower or equal to 100 ppmv, in particular lower or equal to 60 ppmv. The total amount of hydrofluorocarbons potentially remaining in the purified hexafluoro-1,3-butadiene may be equal to or greater than 0 ppmv, 0.001 ppmv, in particular equal to or greater than 0.1 ppmv, in particular equal to or greater than 1 ppmv. It may be measured by conventional methods, such as gas chromatography or mass spectroscopy.

In particular, the total amount of 1,1,4,4-tetrafluoro-1,3-butadiene or potential isomers thereof potentially remaining in the purified hexafluoro-1,3-butadiene may be lower or equal to 50 ppmv, in particular lower or equal to 30 ppmv, in particular lower or equal to 20 ppmv, in particular lower or equal to 10 ppmv, in particular lower or equal to 6 ppmv, The total amount of 1,1,4,4-tetrafluoro-1,3-butadiene and isomers thereof potentially remaining in the purified hexafluoro-1,3-butadiene may be equal to or greater than 0 ppmv, 0.001 ppmv, in particular equal to or greater than 0.1 ppmv, in particular equal to or greater than 1 ppmv. It may be measured by any known method such as gas chromatography or mass spectroscopy.

In a more particular embodiment of the process according to the invention, the at least one first adsorbent is silica gel and the at least on second adsorbent is Chabazite. In terms of sequence, it is preferred that the hexafluoro-1,3-butadiene is first purified by means of at least said silica gel and then by means of at least said Chabazite. Advantageously, the hexafluoro-1,3-butadiene may be purified in a simple an effective way by means of said silica gel subsequently followed by said Chabazite, without requiring any other purification means.

Preferably, the process is conducted at an initial pressure of equal to or above 100 mbar (abs.) and equal to or below 2000 mbar (abs.).

Also preferably, the process is conducted at an initial temperature of equal to or above 5° C. and equal to or below 40° C.

The term “initial” as used herein is intended to denote that temperature and pressure of the gaseous mixture before coming into contact with the primary adsorbent in the sequence comprising at least the first and the second adsorbents.

Also preferably, the flow rate of the gaseous mixture through the adsorbents is set to equal to or above 2 g/min and equal to or below 200 g/min.

In a preferred embodiment, the at least first and second adsorbents are present in different zones in the same adsorber cartridge. Thus, only one adsorber cartridge is used in the purification process and the at least two adsorbents are located within the one cartridge in different zones, preferably in subsequent zones allowing the gaseous mixture to be in contact with one adsorbent after the other.

In another preferred embodiment, the adsorbents that are used in the present invention are present in different adsorber cartridges, so that the gaseous mixture can be brought into contact with the adsorbents one after the other and the adsorbents can be regenerated individually.

According to one embodiment, at least the first adsorbent and/or the second adsorbent, and preferably any of the adsorbents used within the purification process of the invention are not thermally treated before being contacted with the gaseous mixture. Contrary to the purification processes of the state of the art, wherein a pre-treatment often called “activation” which consists in keeping the adsorbent at an elevated temperature, typically between 150 and 400° C., under inert atmosphere to remove moisture from the adsorbent before its first use, the purification process of the invention does not require such a step. It advantageously enables production savings, as it avoids a waste of time, of energy and the management of effluents (mainly water, carbon dioxide and the inert gas used).

The purification process can be repeated as many times as necessary to achieve the desired purity for the final hexafluoro-1,3-butadiene. A recycling loop can therefore be settled to recover the purified hexafluoro-1,3-butadiene downstream of the purification unit and send it back upstream of the purification unit.

The purification process according to the invention may comprise a regeneration step of said at least one first and/or second adsorbent(s). The regeneration step may comprise or consist in a heat treatment of the adsorbent to be regenerated, preferably at a temperature ranging from 200 to 400° C., more preferably from 250 to 350° C., even more preferably from 280 to 300° C. The pressure conditions are not particularly limited: the regeneration step may be advantageously performed at atmospheric pressure.

The hexafluoro-1,3-butadiene purified according to the present invention can be used neat. However, it is often desired to use the hexafluoro-1,3-butadiene of the present invention as an admixture with other fluorinated etching gases to control the carbon/fluoro ratio of the gas mixture. Additionally, mixtures with suitable inert gases like nitrogen, argon or xenon or with oxygen might be desired.

Accordingly, a further aspect of the present invention is a process for the production of a gas mixture according to the present invention, comprising the process for the purification of hexafluoro-1,3-butadiene described above and subsequently, mixing the purified hexafluoro-1,3-butadiene with a further gas selected from the group consisting of an inert gas, oxygen and another fluorinated etching gas as well as the gas mixture formed in such a process.

In particular, one object of the invention is a gas mixture comprising hexafluoro-1,3-butadiene and at least one further gas selected from the group consisting of an inert gas, oxygen and another fluorinated etching gas, wherein the volume ratio of water is less than 200 ppmv and the volume ratio of hydrofluorocarbons is less than 500 ppmv relatively to the total volume of the gas mixture. In particular in said gas mixture, the volume ratio of 1,1,4,4-tetrafluoro-1,3-butadiene or isomers thereof is preferably less than 50 ppmv, relatively to the total volume of the gas mixture.

More particularly, said gas mixture may comprise hexafluoro-1,3-butadiene and at least one further gas selected from the group consisting of an inert gas, oxygen and another fluorinated etching gas, wherein the volume ratio of water is equal to or greater than 0 ppmv and less than 100 ppmv and the volume ratio of hydrofluorocarbons is equal to or greater than 0 ppmv and less than 200 ppmv relatively to the total volume of the gas mixture. In particular in said gas mixture, the volume ratio of 1,1,4,4-tetrafluoro-1,3-butadiene or isomers thereof is preferably equal to or greater than 0 ppmv and less than 20 ppmv, relatively to the total volume of the gas mixture.

The lower limits of the above mentioned impurities may fall in the limit of quantification of the measurement tool. For water the limit of quantification shall appear under 8 ppmv as measured by micro GC. For hydrofluorocarbons, the limit of quantification shall appear under 4 ppm as measured by GC.

The inventive gas mixtures can easily be prepared by condensing or pressing the desired amounts of hexafluoro-1,3-butadiene and any other desired gas into a pressure bottle.

Furthermore, the invention concerns a process for the production of a semiconductor material, a solar panel, a flat panel or a microelectromechanical system, or a process for cleaning the chamber of an apparatus used for semiconductor manufacturing using the hexafluoro-1,3-butadiene purified according to this invention or the gas mixture according to this invention. The preferred use is in the production of a microelectromechanical system.

Should the disclosure of any patents, patent applications, and publications which are incorporated herein by reference conflict with the description of the present application to the extent that it may render a term unclear, the present description shall take precedence.

FIG. 1 shows a suitable apparatus for the process of the present invention. Initial tank C1 contains the crude hexafluoro-1,3-butadiene. The amount of hexafluoro-1,3-butadiene in tank C1 can be measured by means of a balance. Final tank C2 is submersed in a cooling bath at −78° C. (mix of dry ice and acetone). Stainless steel tube A1 contains the adsorbent beds. It has an internal diameter of 18 mm and a length of 406 mm. It is double jacketed and connected to a cooling bath for being able to cool down the bed in case of an exothermic reaction inside. Pressure and temperature of the gaseous mixture before and after tube A1 are measured. All piping is made of stainless steel.

The following describes a typical sequence of the inventive process using the apparatus as shown in FIG. 1.

None of the adsorbents are pre-treated before use: they are directly loaded into tube A1 or stored for later usage.

Once tube A1 is charged with the required adsorbents, it is installed in the apparatus and the tightness of the apparatus is checked under vacuum.

Afterwards, final tank C2 is submersed into the cooling bath and tank C1 is charged with 2500 g of crude hexafluoro-1,3-butadiene. The pressure in tank C1 is usually in the range from 1.5 bar to 1.8 bar (abs.).

The crude hexafluoro-1,3-butadiene is then allowed to pass through tube A1 and the thus purified hexafluoro-1,3-butadiene is collected by condensation in final tank C2. The flow is manually controlled from 5 g/min to 25 g/min by adjusting needle valves V1, V2 and V3 accordingly.

After all crude hexafluoro-1,3-butadiene has been passed through tube A1, tank C2 is isolated by closing valve V4 and then allowed to warm to room temperature.

A sample of the purified hexafluoro-1,3-butadiene in tank C2 is analyzed and the analysis results are compared to those of the crude hexafluoro-1,3-butadiene.

The following example shall explain the invention in further details, but is not intended to limit the scope of the invention.

### Example 1: Purification of Hexafluoro-1,3-butadiene Using a Combination of Silica Gel and Chabazite

For this trial, tube A1 was first charged with 1300 g silica gel Sylobead SG B 125 supplied by GRACE, average pore size of 12.5 Å), without any pre-treatment, at the end of tube A1 which comes first into contact with the gaseous mixture. Afterwards, the rest of tube A1 was charged with 1300 g of Chabazite HCZC S (H-form) supplied by CLARIANT, average pore size of 3.8 Å), without any pre-treatment, at the end of the tube A1 facing final tank C2. Thus, tube A1 was charged with two separate adsorber beds, a first bed with silica gel and a subsequent bed with Chabazite. Following the typical procedure described above, an initial amount of 2500 g hexafluoro-1,3-butadiene was purified at a flow rate of 5 g/min, a pressure measured at pressure gauge P2 of 1.2 bar (abs.) and a temperature of 10° C. measured at thermocouple T2.

The results of the analyses of the crude hexafluoro-1,3-butadiene from tank C1 and the purified hexafluoro-1,3-butadiene from tank C2 are shown in table 1.

TABLE 1
Analysis results

| impurites (ppmv) | | |
|---|---|---|
| relative to the total hexafluoro-1,3-butadiene | Tank C1 | Tank C2 |
| water | <1000 | <8 |
| C4H2F4 | <200 | <5 |
| Total hydrofluorocarbons (ppmv) | <1500 | <50 |

Some of the results were in the limit of quantification of the measurement tool: this is the case of water, for which the limit of quantification appears under 8 ppmv as measured by micro GC. The C4H2F4 and total HFCs were quantified by GC.

1. A process for the purification of hexafluoro-1,3-butadiene comprising a step wherein a gaseous mixture comprising hexafluoro-1,3-butadiene is contacted with at least one first adsorbent and at least one second adsorbent to purify said gaseous mixture, wherein the at least one first adsorbent has an average pore size of more than 10 Å and the at least one second adsorbent has an average pore size of less than 4 Å.

2. The process according to claim 1, wherein the at least one first adsorbent has an average pore size of more than 10 Å and less than 100 Å.

3. The process according to claim 1, wherein the at least one second adsorbent has an average pore size of more than 1 Å and less than 4 Å.

4. The process according to claim 1, wherein the at least one first adsorbent is suitable to remove at least water molecules.

5. The process according to claim 1, wherein the at least one second adsorbent is suitable to remove at least one impurity selected from hydrohalogenocarbons.

6. The process according to claim 1, wherein the gaseous mixture is first purified with the at least one first adsorbent and subsequently purified with the at least one second adsorbent.

7. The process according to claim 1, wherein the gaseous mixture is contacted with the at least one first adsorbent and the at least one second adsorbent at an initial pressure of equal to or above 100 mbar (abs.) and equal to or below 2000 mbar (abs.).

8. The process according to claim 1, wherein the gaseous mixture is contacted with the at least one first adsorbent and the at least one second adsorbent at an initial temperature of equal to or above 5° C. and equal to or below 40° C.

9. The process according to claim 1, wherein the gaseous mixture is contacted with the at least one first adsorbent and the at least one second adsorbent at a flow rate of equal to or above 2 g/min and equal to or below 200 g/min.

10. The process according to claim 1, wherein the at least one first adsorbent and the at least one second adsorbent are present in different zones within one adsorber cartridge.

11. The process according to claim 1, wherein the at least one first adsorbent and the at least one second adsorbent are present in two different adsorber cartridges.

12. The process according to claim 1, wherein the at least one first adsorbent and/or the at least one second adsorbent are not thermally treated before being contacted with the gaseous mixture.

13. A process for the production of a gas mixture comprising the process according to claim 1 and subsequently, mixing a purified hexafluoro-1,3-butadiene with a further gas selected from the group consisting of an inert gas, oxygen and another fluorinated etching gas.

14. A gas mixture comprising hexafluoro-1,3-butadiene and at least one further gas selected from the group consisting of an inert gas, oxygen and another fluorinated etching gas, wherein the volume ratio of water possibly contained therein is less than 200 ppmv and the volume ratio of hydrofluorocarbons possibly contained therein is less than 500 ppmv, relatively to the total volume of the gas mixture.

15. A process for the production of a semiconductor material, a solar panel, a flat panel or a microelectromechanical system, or a process for cleaning the chamber of an apparatus used for semiconductor manufacturing, comprising producing the semiconductor material, solar panel, flat panel or microelectromechanical system or cleaning the chamber with the hexafluoro-1,3-butadiene purified according to the claim 1 with a gas mixture comprising hexafluoro-1,3-butadiene and at least one further gas selected from the group consisting of an inert gas, oxygen and another fluorinated etching gas, wherein the volume ratio of water possibly contained therein is less than 200 ppmv and the volume ratio of hydrofluorocarbons possibly contained therein is less than 500 ppmv, relatively to the total volume of the gas mixture.
